(19) United States
(12) Patent Application Publication
(10) Pub. No.: US 2002/0128714 A1
(43) Pub. Date: Sep. 12, 2002

(54) Orthopedic implant and method of making metal articles

(76) Inventors: Mark Manasas, Dedham, MA (US); Keith E. Oslakovic, Cambridge, MA (US); Cornel Sultan, Everett, MA (US); John V. Hamilton, Foxboro, MA (US); Donald E. Ingber, Boston, MA (US); Carmine Sammarco, Medfield, MA (US); John Kummailil, Framingham, MA (US); David J. Skinner, Mansfield, MA (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 09/874,712

(22) Filed: Jun. 5, 2001

Related U.S. Application Data
(63) Continuation-in-part of application No. 09/588,167, filed on Jun. 5, 2000.
(60) Provisional application No. 60/291,183, filed on May 15, 2001.
(60) Provisional application No. 60/137,626, filed on Jun. 4, 1999.

(51) Int. Cl. A61F 2/44

(52) U.S. Cl. 623/17.15

(57) ABSTRACT

The present application is directed to an orthopedic implant. More specifically, the orthopedic implant is suitable for arthroplasty procedures where optimized multifunctional behavior of the implant is desired. In some embodiments the implant is suitable for the replacement of a spinal disc. In one embodiment, the present application is directed to an orthopedic implant including a first plate a second plate and a flexible support. The flexible support may have a single connection to the first plate and a single connection to the second plate and may vary in cross section. The first plate, the second plate and the flexible support may be unitarily formed.

This application is also directed to methods of producing metal articles having microstructure for improved mechanical properties. Such methods may be suitable for the production of medical devices. In one embodiment, the method includes directing a stream including a particulate material in a pattern corresponding to at least a portion of a structure of an orthopedic implant and fusing at least a portion of the particulate material with a laser.

This patent application claims priority to U.S. patent application Ser. No. 09/588,167, filed Jun. 5, 2000, and to U.S. Provisional Patent Application No. 60/291,183, filed May 15, 2001.

### BACKGROUND

1. Field

This application is directed to an orthopedic implant. More specifically, the orthopedic implant is suitable for arthroplasty procedures where optimized multifunctional behavior of the implant is desired. In some embodiments the implant is suitable for the replacement of a spinal disc. This application is also directed to methods of producing metal articles having microstructure for improved mechanical properties. Such methods may be suitable for the production of medical devices.

2. Description of the Related Art

Orthopedic implants have been used to repair damage to the skeleton and related structures, and to restore mobility and function. For example, various devices, such as pins, rods, surgical mesh and screws, have been used to join fractured bones in a proper orientation for repair.

Implants that restore function to a damaged joint have also been used. Surgery intended to restore function to a joint is referred to as arthroplasty. A successful arthroplasty may eliminate pain and prevent the degradation of adjacent tissue. Arthroplasty has been performed on knees, hips and shoulders by replacing portions of the joint with implants.

One issue with presently available implants for arthroplasty is that they may result in stress shielding, meaning that a stress normally felt by bone adjacent to the implant is reduced due to the stiffness of the implant. When a bone is shielded from physiologic loads, it typically reduces in size and strength according to Wolff's Law, thereby increasing the chance of its breakage.

In some instances, instead of replacing a damaged joint, the joint is merely fused in a single position. Surgery intended to fuse a joint rather than to restore mobility is referred to as arthrodesis. Arthrodesis is particularly common for the complex load-bearing joints of the spine. Spinal fusion may be performed to remedy failure of a spinal disc.

Spinal discs perform spacing, articulation, and cushioning functions between the vertebrae on either side of the disc. If the normal properties of a disc are compromised, these functions can be seriously reduced. Disc collapse or narrowing reduces the space between vertebrae, and damage to the disc can cause it to bulge or rupture, possibly extruding into the spinal canal or neural foramen. These changes can cause debilitating pain, numbness, or weakness.

Orthopedic implants may be used in arthrodesis to stabilize the spine and promote fusion. The two main surgical approaches to implant-aided spinal fusion are anterior and posterior. Anterior fusion techniques are widely used, primarily due to Interbody Fusion Devices (IBFDs). IBFDs are inserted into the space normally occupied by the disc to restore disc height and stabilize the spine. Posterior fusion is accomplished by exposing the spinal segments through the musculature of the back and fixing adjacent vertebra using hardware typically consisting of metal rods, screws and other devices. Bone harvested from the patient's iliac crest (autograft), donor bone (allograft), or other synthetic biocompatible material is sometimes also packed into the space to induce fusion.

U.S. Pat. No. 5,860,973 (hereinafter “Michelson”) discloses an implant that is placed translaterally between two discs. The implant, which is typically installed as a pair of implants, is cylindrical and is filled with fusion promoting material. During the installation, holes are bored between the vertebra and the implant is placed within the holes. The fusion material solidifies into bone, thus fusing the adjacent vertebrae together.

Another way to treat spinal damage is to replace the damaged vertebra or disc with a spacer. For example U.S. Pat. No. 5,702,451 (hereinafter “Biedermann”) discloses a space holder for a vertebra or spinal disc consisting of a hollow sleeve perforated with diamond-shaped holes. The holes are sized and arranged such that when different lengths of sleeve are cut, the recesses along the edge of the cut resulting from the diamond shaped holes are uniform. If desired, an end cap may be mated with the resulting projections on the end of the sleeve. The cut end of the sleeve, or the attached end caps, are then positioned in apposition to the vertebral endplates.

Both spinal fusion, such as disclosed by Michelson, and the use of spacers, such as disclosed by Biedermann, limit the mobility of the spine by fixing two adjacent vertebrae relative to one another. In addition to reduced mobility, these arrangements do not compensate for the shock absorption lost when a disc is damaged or removed.

Attempts to restore lost function to damaged spinal joints (arthroplasty) have also been made. For example, replacement of entire discs or simply the nucleus pulposus (center portion of the disc) have been proposed. Some attempts use elastomers to mimic the shock absorption and flexibility of the natural disc. However, the complex load bearing behavior of a disc has not been successfully reproduced with an elastomer, and such implants are prone to wear and failure. For example, U.S. Pat. No. 5,674,294 (hereinafter “Bainville”) describes an intervertebral disc spacer having two metal half-envelopes that confine between them a cushion. Similarly, implants using various liquids and gels have also been attempted. These implants are subject to failure by rupture or drying out, just like a disc. Mechanical approaches to disc replacement have also been attempted. For example, articulating surfaces and spring-based structures have been proposed. In addition to failing to accurately perform the functions of the replaced disc, these structures are multi-component and particles generated by wear of articulating components can result in adverse biological responses or increase the possibility of mechanical failure. One example of a multi-component structure is disclosed by U.S. Pat. No. 5,893,889 (hereinafter “Harrington”) which describes an artificial disc having upper and lower members joined by a pivot ball and having shock absorbing members fitted between the upper and lower member.

The most successful arthroplasty procedures have been total hip arthroplasties. Total hip arthroplasty devices using rigid structures as the load sharing devices between the femur and the acetabulum have also been observed to experience the phenomena referred to as stress shielding. In the case of a hip stem, the method of load transfer is typically changed with the insertion of the implant. In the normal femur, the loads are applied to the femoral head and transferred along the length of the femur through the cortical shell of the femur. It is difficult to match the both the proximal and distal geometry of an implant to its host bone. In the case of the femur with an implant, the implant frequently subsides until the distal geometry becomes wedged in the metaphyseal canal. The prosthesis therefore channels the loads distally down the prosthesis and loads the inside of the metaphyseal cortical shell. This results in a significant portion of the proximal bone of the femur no longer experiencing a normal stress condition. This condition may result in a loss of bone mass surrounding the proximal portion of the device. Consequences of this bone loss include reduced proximal support for the device, which will allow the device to move and become painful. Consequently, should revision of the device be required, there may be insufficient bone for support of the revision implant.

A number of approaches have been attempted to solve this problem. These include use of composite materials for controlled stiffness of the bulk material, modifications of the cross section of the device to reduce stiffness (this includes local reduction in cross section and hollow stems) and incorporation of slits in the device to increase flexibility. None of these approaches have been successful in that the compromises required to achieve the reduction in stiffness did not allow the required strength.

The knee joint has also been the subject of arthroplasty procedures. In total knee arthroplasties, one of the major clinical issues is wear between the femoral and tibial articulating surfaces. These wear surfaces are typically made up of two dissimilar materials, commonly a polymer and a metal. Typically, ultra high molecular weight polyethylene (UHMWPE) is used as the polymer. While this material has excellent wear properties, it is not a wear free surface. The cartilage of a normal knee is not a bulk tissue but has an internal structure that allows the generation of a fluid film on the articulating surface upon the application of physiological loads and motions. This fluid film is then used as a lubricant to reduce the coefficient of friction between the two cartilage wear surfaces. However, there is no fluid film lubricant in the total knee joint implants presently used. Instead, the materials articulate directly on one other resulting in the generation of wear debris, which may produce adverse biological responses.

Several attempts have been made to incorporate stochastic foam materials to reproduce this fluid film lubrication mechanism in the knee joint. None of these approaches, however, have been successful in reproducing the functionally graded material properties required for this application.

### SUMMARY

In one embodiment, the present application is directed to an orthopedic implant including a first plate, a second plate and a flexible support having a single connection to the first plate and a single connection to the second plate. In this embodiment, the first plate, the second plate and the flexible support are unitarily formed.

In another embodiment, the present application is directed to an orthopedic implant including a first plate, a second plate and a flexible support. The flexible support includes a connection to the first plate, a connection to the second plate and a cross section varying along a length of the flexible support.

In another embodiment, the present application is directed to an orthopedic implant produced by a method including directing a stream including particulate material in a pattern corresponding to at least a portion of a structure of the orthopedic implant and fusing at least a portion of the particulate material with a laser.

In another embodiment, the present application is directed to an article including a metallic component comprising layers including grains of precipitated phase wherein a majority of the grains of precipitated phase in each layer are oriented in substantially two opposed directions and the two opposed directions are substantially different from a direction of orientation of grains in an adjacent layer.

In another embodiment, the present application is directed to an orthopedic implant including an outer wall and a flexible support connected to the outer wall in at least two locations.

In another embodiment, the present application is directed to a method of making an orthopedic implant. The method includes directing a stream including a particulate material in a pattern corresponding to at least a portion of a structure of the orthopedic implant and fusing at least a portion of the particulate material with a laser.

In another embodiment, the present application is directed to a method of making an article. The method includes directing a stream of a particulate material at a rate greater than about 4 grams per minute and less than about 20 grams per minute and moving the stream in a pattern corresponding to at least a portion of a structure of the article at a velocity greater than about 50 centimeters per minute and less than about 250 centimeters per minute. The method further includes fusing at least a portion of the particulate material with a laser having a power greater than about 100 joules per second and less than about 600 joules per second.

In another embodiment, the present application is directed to a method of making an article. The method includes directing a stream including a particulate material, moving the stream in a first pattern corresponding to at least a first portion of a structure of the article to form a first layer of the article and fusing at least a portion of the first pattern with a laser. The method further includes moving the stream in a second pattern corresponding to at least a second portion of the structure of the article to form a second layer of the article, wherein the second pattern is displaced between one of 1 to 89 and 91 to 179 degrees from the first pattern and fusing at least a portion of the second pattern with a laser.

### BRIEF DESCRIPTION OF THE DRAWINGS

Preferred, non-limiting embodiments of the present application will be described by way of example with reference to the accompanying drawings, in which:

FIG. 1 is a perspective view of one embodiment of an orthopedic implant of this application;

FIG. 2 is a perspective view of another embodiment of an orthopedic implant of this application;

FIG. 3 is a perspective view of another embodiment of an orthopedic implant of this application;

FIG. 4 is a perspective view of another embodiment of an orthopedic implant of this application;

FIG. 5 is a schematic, top view of one aspect of a method of making an article of this application;

FIG. 6 is a schematic, top view of another aspect of a method of making an article of this application;

FIG. 7 is a schematic, top view of another aspect of a method of making an article of this application;

FIG. 8 is a schematic, top view of another aspect of a method of making an article of this application;

FIG. 9 is a schematic, top view of another aspect of a method of making an article of this application;

FIG. 10 is a photocopy of a photomicrograph of one aspect of an article of this application;

FIG. 11 is a photocopy of a photomicrograph of another aspect of an article of this application;

FIG. 12 is a photocopy of a photomicrograph of another aspect of an article of this application;

FIG. 13 is a graph of a correlation between build parameters (Φ) versus build height (δ) according to a method of making an article of this application;

FIG. 14 is a front and side schematic view of another embodiment of an orthopedic implant of this application;

FIG. 15 is a cross-sectional view of one aspect of the embodiment of FIG. 14;

FIG. 16 is a front and side schematic view of another embodiment of an orthopedic implant of this application;

FIG. 17 is a graph representing the width and thickness of one aspect of the embodiment of FIG. 16;

FIG. 18 is a schematic representation of one aspect of the embodiment of FIG. 16;

FIG. 19 is a perspective view of another embodiment of an orthopedic implant of this application;

FIGS. 20a and 20b are two graphs representing the width and thickness of one aspect of the embodiment of FIG. 19;

FIG. 21 is a schematic representation of one aspect of the embodiment of FIG. 19;

FIG. 22 is a perspective, cut-away view of another embodiment of an orthopedic implant of this application;

FIG. 23 is a perspective view of another embodiment of an orthopedic implant of this application;

FIG. 24 is a perspective view of another embodiment of an orthopedic implant of this application;

FIG. 25 is a graph of maximum fatigue load and maximum flexion for three different embodiments of this application;

FIG. 26 is a perspective, cut-away view of an aspect of an article of manufacture of this application;

FIG. 27 is a side, cut-away view of the aspect illustrated in FIG. 26;

FIG. 28 is a perspective, cut-away view of an aspect of an article of manufacture of this application;

FIG. 29 is a side, cut-away view of the aspect illustrated in FIG. 28; and

FIG. 30 is a perspective, cut-away view of one aspect of the present application.

### DETAILED DESCRIPTION

The design of an optimized implant for use in arthroplasty, such as spinal disc replacement, may be done by several methods, including using functionally adapted software, such as that described in U.S. patent application Ser. No. 09/400,516, titled “METHOD AND APPARATUS FOR DESIGNING FUNCTIONALLY ADAPTED STRUCTURES HAVING PRESCRIBED PROPERTIES,” which is herein incorporated by reference. This design methodology involves the use of a seed geometry that has been screened for a prescribed set of mechanical properties. The seed geometry may be adjusted to fill a design envelope defined as the space available for an implant as determined from anatomical studies. A set of inputs to the methodology may then be determined and provided, the inputs representing the functional requirements for a clinically successful implant. The next step may be to optimize the seed geometry using the functionally adapted software, such as that described in the above-mentioned U.S. Patent Application. In the case of a lumbar spinal disc replacement, loading conditions and corresponding stiffness requirements may be used as the input conditions for the optimization algorithm. Additional criteria also may be applied, such as maintaining peak stresses at a stress level at or below the fatigue endurance limit of the material the implant is to be fabricated from.

In addition to the functionally adapted software mentioned above, there are several other approaches that can be taken to optimize the structure. For example, commercially available software packages such as Pro Engineer produced by Parametric Technologies Corporation of Waltham, Mass. and ANSYS produced by ANSYS, Inc. of Canonsburg, Pa. may be used to optimize some parameters based on, for example, geometric considerations, material properties and the functional properties of the joint to be replaced.

The seed geometries used for the optimization method may be taken from a library of three dimensional geometries such as those described in the above-referenced U.S. patent application. Examples of these geometries are structures such as octet trusses and kelvin foams. These seed geometries may be combined in a continuous or discontinuous manner. The combination of these geometries is known as combinatorial geodesics.

The seed geometries need not be homogeneous. Instead, for example, if anisotropic properties are desired, the seed geometries may be adjusted such that these are also anisotropic. Thus, an implant may include differing seed geometries. These seed geometries may be solid, porous or other standard manufacturing constructs such as braids, woven materials or laminates. Furthermore, the seed geometries are not required to be constant in cross section, instead, the geometric properties of the cross section may be varied throughout the structure.

One feature to the design of implants using the techniques described above is the recognition that designs do not have to be limited to the traditional manufacturing constraints such as those imposed by conventional machining or casting methods. These methods have limitations regarding the size and shape of the features that may be produced. Construction of implants designed using the techniques described above may be with the use of other manufacturing techniques such as solid free form fabrication. Some examples of solid free form fabrication include, but are not limited to, directed deposition of metals (also known as Laser Engineered Net Shape [LENS] methods), Selective Laser Sintering (SLS) and 3D printing. All of these approaches may be used in combination with a Hot Isostatic Pressing (HIP) method to produce a product substantially free of internal porosity and defects. The LENS method includes directing a stream of metal powder into a mobile laser which melts the metal. As the laser moves, the metal solidifies. Subsequent layers of metal may be deposited on one another, allowing a three dimensional structure to be built up. The LENS method and other laser deposition-related methods that may find applicability to orthopedic implants are described more fully in U.S. Pat. No. 4,323,756 to Brown et al., U.S. Pat. No. 4,724,299 to Hammeke et al., U.S. Pat. No. 5,043,548 to Whitney et al., U.S. Pat. No. 5,578,227 to Rabinovich, U.S. Pat. Nos. 5,837,960 and 5,961,862 to Lewis et al., U.S. Pat. No. 5,993,554 to Keicher et al. and U.S. Pat. No. 6,046,426 to Jeantette et al., and these patents are hereby incorporated by reference. Combinations of these manufacturing techniques and the optimization approaches described above allow for the design, optimization and manufacture of novel structures with multifunctional features according to this application. In particular, according to one aspect of this application, there are provided novel implant structures, such as unitary structures, that have significant variations in stiffness in the axial and flexion/extension orientations.

In one aspect, the present application is directed to a method and system for the production of simple and/or complex shaped metal articles that are imparted with superior mechanical properties to that normally associated with standard production methods for the metal, such as casting and wrought techniques. As used herein, “metal” includes both pure metals and metal alloys. A metal may include natural alloys, man made alloys and metals with non-metal additives and still fall within this definition.

Known methods of producing solid articles include laser deposition, such as the LENS (Laser Engineered Net Shape) method, as discussed above. However, there has been no definition of system running parameters, real or implied, that effect a microstructure of an article produced by such a method, in order to enhance specific mechanical properties of the article. It should be understood that while this description provides one theory describing a cause of the improved properties observed in accordance with the articles and method of the present application, the present application should not be construed as being limited to any particular theory or cause, and instead is defined by the scope of the claims.

One aspect of this application describes setup and running parameters of a method that allows tailoring and/or refinement of a microstructure of metals, improving the mechanical properties of the metals, particularly for stress, fatigue, cracking, and the like. Such a method and the resultant metals may be suitable for medical devices, such as implants, which may require excellent mechanical properties.

In conventional metal powder deposition techniques, such as the LENS method, as described in U.S. Pat. No 6,046,426 to Jeantette et al., a controlled stream of powdered metal is directed into a focused laser beam, is melted and thereby deposited onto a substrate. According to one aspect of this disclosure, the LENS method can use a powdered metal, such as titanium alloy of suitable grade for medical devices. The movement of the stream of metal powder is controlled in order to produce a solid article of either simple or complex shape. The solid article is formed by the deposition of multiple layers. Each layer represents a combination of single linear powdered metal/laser beam interactions in a systematic overlapping pattern, such as in a hatch design, as illustrated in FIGS. 5 and 6. Multiple layers thus applied produce a solid article either of a desired net shape, or near net shape, of about 100% density. In alternate embodiments, instead of using a powdered metal, a continuous wire of metal may be melted in the desired pattern using the laser. Though a laser is described herein, any energy source capable of melting the metal may be used. For example, the laser could be a Nd:YAG Laser, a CO laser, a CO₂ laser, another laser type or even another heat source, such as a MIG (Metal-Inert Gas) or TIG (Tungsten-Inert Gas) welder.

Each layer laid down by the metal powder deposition method is typically comprised of an outer perimeter line and a “hatch” design, as described above, for space filling, such that each layer represents a continuous slice of material approaching 100% density. It should be appreciated that the speed at which the laser and powdered metal stream is moved may be different for the outer perimeter and the hatch design. For example, the outer perimeter may be formed at the same or faster rate than the hatch design that fills it.

A single layer may be started in any location and laid down across the substrate. Typically, a start point for each layer is a corner, improving continuity of the hatch design. One example hatch pattern for a single layer is illustrated in FIG. 5. A second layer is typically added to the first layer in a hatch pattern 90° offset from the hatch pattern of the first layer, as illustrated in FIG. 6. FIGS. 5 and 6 thus illustrate the motion of a linear, powdered metal/laser beam interaction from its start position 100 for each layer as it fabricates two sequential layers in the production of an article 10. Additional layers may be built upon the first two layers. A suitable start position 100 of the laser beam in producing each layer of a simple shaped article in a systematic rotational procedure, is thus any one of the four corners as illustrated in FIG. 7.

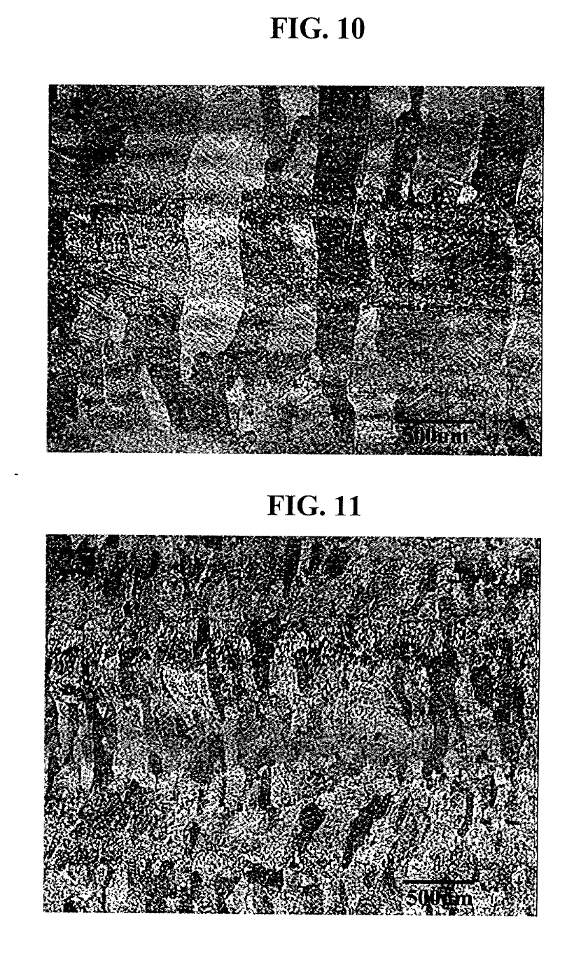

Illustrated in FIG. 10 is one example embodiment of a titanium alloy, wherein epitaxial growth of columnar β titanium alloy grains during deposition of subsequent layers results from the partial remelting of the previous layer. The heat conduction path through the article being manufactured promotes the epitaxial growth of existing β titanium alloy grains into the receding melt pool. This results in an average β titanium alloy grain length of approximately 2000 μm and an average β titanium alloy grain width of approximately 300 μm. This sequence of grain growth is expected when the method of manufacture parameters for the LENS process are such that the melt zone from the linear, powdered titanium alloy/laser beam interactions supplies enough energy to cause the liquid titanium alloy to experience a high degree of superheat (for example >200 K). As used herein, “superheat” refers to the degree by which the temperature of the liquid metal exceeds the normal melting temperature of the metal. The higher degree of superheat increases the remelting of the previous layer, encouraging β titanium alloy grain growth.

As illustrated in FIG. 11, by a judicious choice of values for laser power, velocity of motion of the linear powdered metal/laser beam interaction, and powder deposition rate, as will be described below in greater detail, the superheat described above for the melt pool is reduced to a value that promotes the nucleation of β titanium alloy grains within the melt pool itself, thereby producing a microstructure whereby the columnar β titanium alloy grains are defined by, at most, two subsequent layers of the build, (approximately 300 μm in average length and 100 μm in average width, though these values may vary widely based on specific conditions).

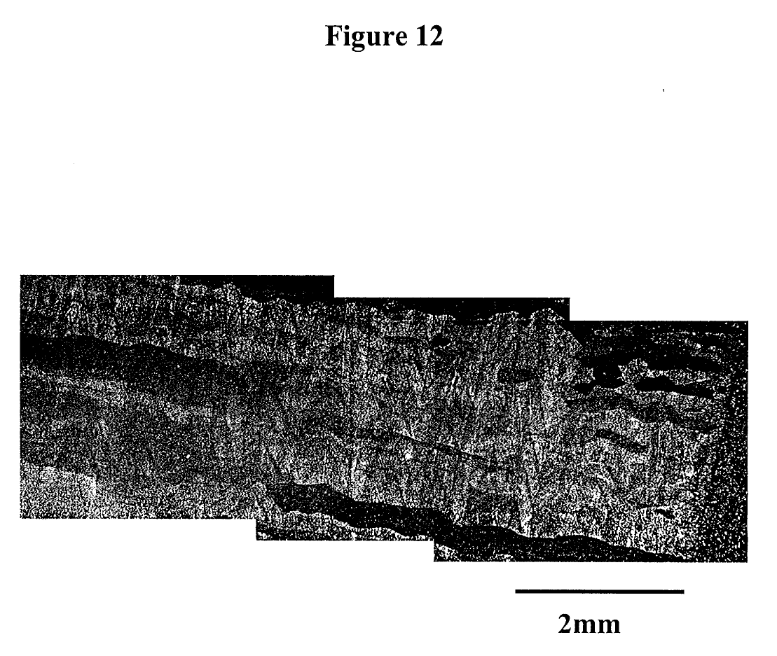

FIG. 12 is a photocopy of a photomicrograph of an article after conventional hot isostatic pressing (HIP) and heat treatment procedures illustrating (a) β titanium alloy grains normal to the direction of layer build and (b) the directionality of α precipitation within each layer. FIG. 30 shows this structure in three dimensions, with the direction of the laser motion that produced the structure illustrated by arrows 103. This directional α precipitation within each layer is the result of the solidification profile caused by the movement of the powdered metal/laser beam interactions and controlled parameters with their attendant smaller melt pool. The directionality produces a microstructure that may be described as herringbone or tweed. It should be appreciated that where each layer is built in a hatch pattern the α precipitation will occur in rows within each layer having opposite orientations in accordance with the motion of the laser.

The reduction in prior β titanium alloy grain size brought about through the refinement of method parameters, as described above, will bring about a benefit of improved fatigue initiation resistance, a physical property valuable in many fields and particularly pertinent to the area of medical devices that undergo bending or rotation once implanted in the body. As a consequence of the layered structure and cooling rate directionality, orientation of the α phase precipitation during the HIP and heat treatment procedures optimizes mechanical properties of an article and thus of any medical device manufactured through this technique. Articles made by this method also exhibit improved fatigue crack growth resistance due to the more tortuous path(s) necessary for any crack to grow (cracks tend to follow interfaces within a microstructure). Because of the “tweed” nature of the α precipitates, any crack will be forced to change direction as the α precipitate direction changes within the microstructure. Furthermore, the nature of the α precipitation produces a multi-modal distribution of size and orientation (of these α precipitates), improving strength, ductility and fatigue initiation resistance.

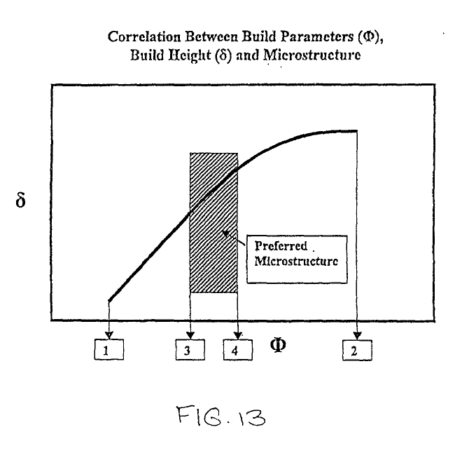

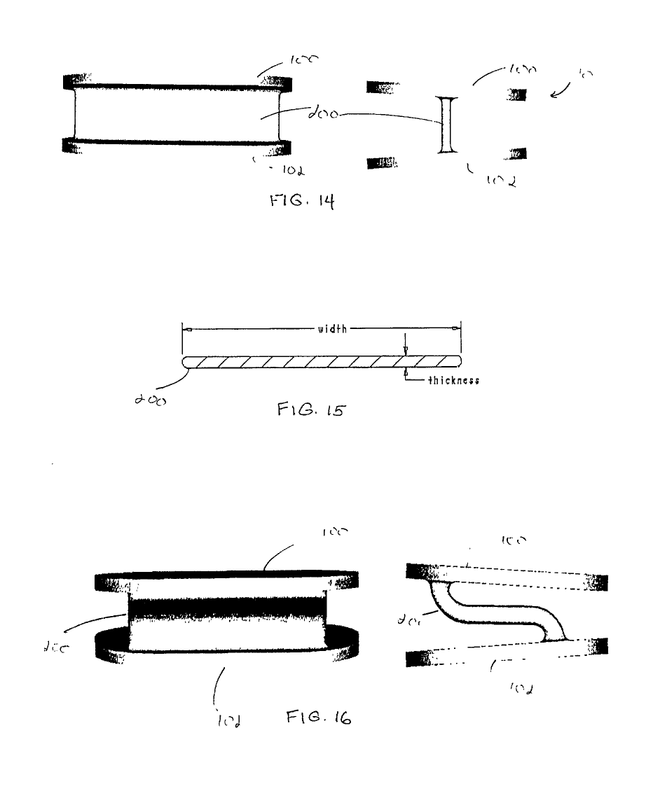

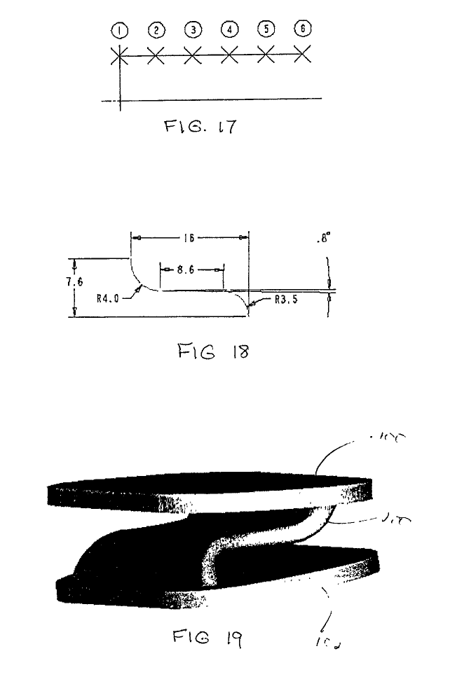

FIG. 13 is a graph representing correlation between build parameters (Φ), average layer thickness (δ) and microstructure. Φ is defined by the build parameters, such as laser power, speed of motion of laser and the rate powder is supplied to the focused laser beam. Φ may be expressed as:

Φ=(Tₒₙ.P_{L}.m)V(J.kg.m⁻³.s⁻¹) (1)

where Tₒₙ is the time the laser beam is on during building an article of the invention in seconds and P_{L} is the laser power in J/s. Tₒₙ is provided through the machine software controlling the build parameters. Thus, the quantity Tₒₙ is a function of the speed of laser beam and also the volume of the part to be constructed. m is the powder feed rate supplied to the focused laser beam in kg.s⁻¹. V is the volume of the part to be constructed in m³.

The quantity Φ can be correlated with a degree of certainty to the average build height (δ), i.e., the thickness of each deposited layer built under these conditions. Values of Φ between positions 1 and 2 on FIG. 13 provide acceptable build height (δ). Values of Φ between positions 3 and 4 provide a preferred range of values for Φ that achieves the desired refined microstructure. Solid articles can be produced by selecting values of laser power, travel velocity of laser beam and amount of powder arriving at the focused laser beam per unit time, with the dimensions of the article to be built (thus calculating a value for Φ, between position 1 and 2 in FIG. 13) and thus determine the build height (δ). Height of the article is specified by producing height divided by δ layers.

The preferred values for Φ to achieve the desired refinement of the β titanium alloy grain size, as described above, and its consequential improvements in properties are shown in FIG. 13, (between the positions 3 and 4). For example, for an article 0.0127 m×0.0127 m×0.019 m constructed of a titanium alloy containing 6% aluminum and 4% vanadium, it has been found that values for Φ of between about 9.5×10⁶ and about 11×10⁶ are in the preferred range of microstructure while values of Φ of between about 5.0×10⁶ and about 20×10⁶ provide acceptable build height (δ). It should be appreciated, however, that these values are highly dependent upon the metal and the dimensions of the article being constructed and are intended by way of illustration only.

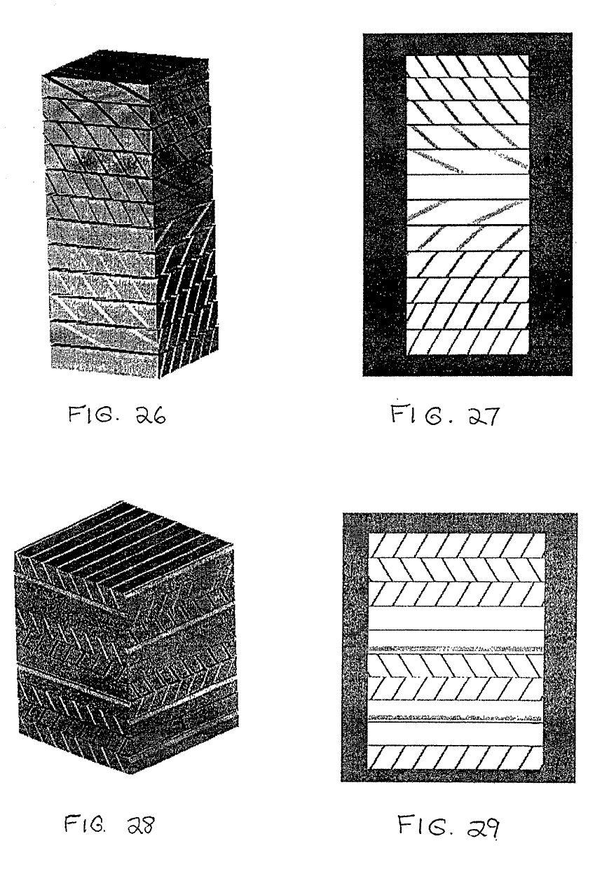

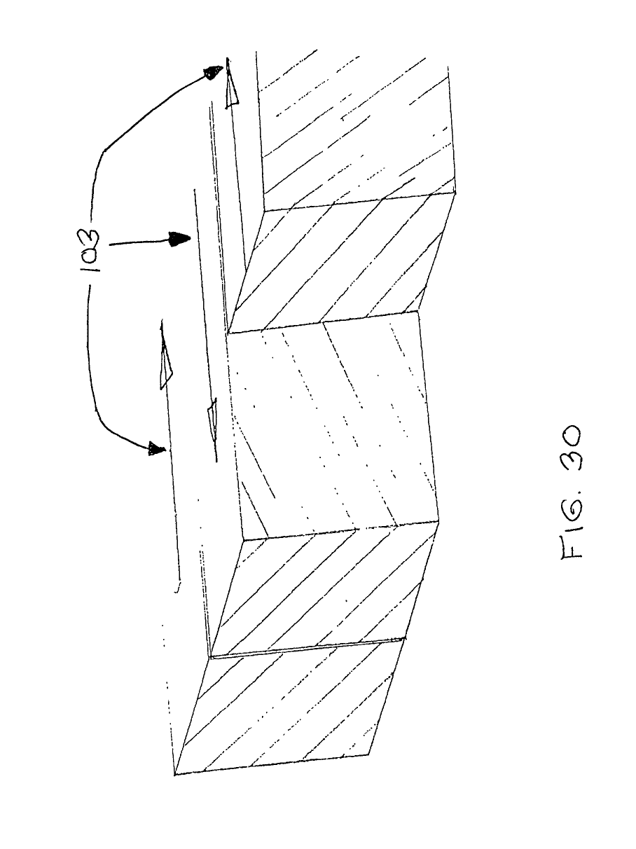

It has been discovered that by modifying the starting point for each layer and the resulting angle θ between subsequent layers from 90°, as illustrated in FIGS. 8 and 9, the microstructure an article can be further controlled. An example of this microstructure is illustrated in FIGS. 26 and 27, which illustrate an article where each layer varies 15° from the previous layer. This is in contrast to a traditional 90° variation, as illustrated in FIGS. 28-29. It is to be appreciated that while FIGS. 28 and 29 do not illustrate layers varying from 90°, they do illustrate the layered microstructure of the application, as described above. It is believed that by varying the angle of each layer from 90° with respect to its neighbors, that the path necessary for crack propagation will be made more tortuous and the resistance to cracking of a resultant article will be increased. Rotational symmetry of the microstructure may also be achieved in such a method where the layers are not orthogonal (θ≈90°). For example, where the layers are disposed by 105°, rotational symmetry is achieved in 24 layers, where they are disposed by 120°, rotational symmetry is achieved in 12 layers, and where the layers are disposed by 135°, rotational symmetry is achieved in 8 layers.

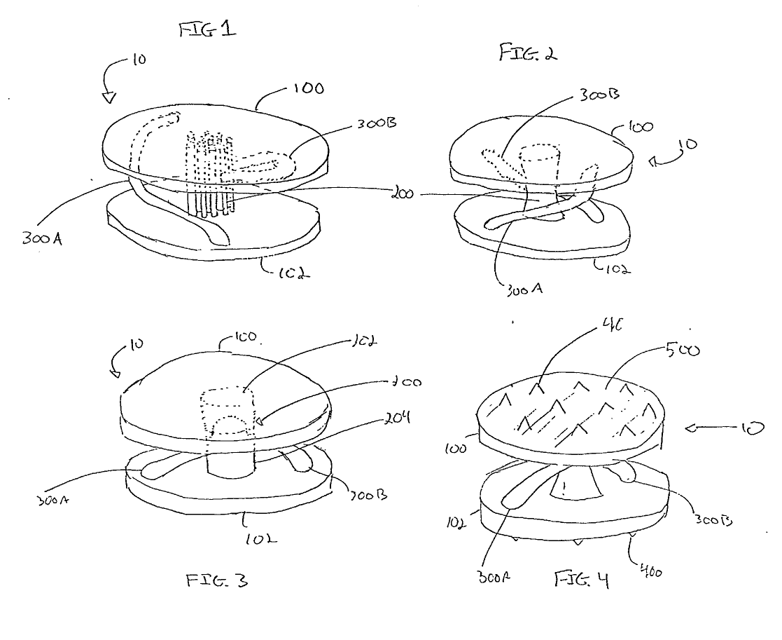

Referring now to FIG. 1, one example of an orthopedic implant according to an embodiment of the application is illustrated. Orthopedic implant 10 includes a first plate 100, a second plate 102, an axial support 200 between plates 100, 102 and one or more torsional supports 300A, 300B connecting the first plate and the second plate. As used herein, the axial means along an axis that is substantially perpendicular to the primary surfaces of plates 100, 102 and an axial support is a structure that provides support and resistance to compression in the axial direction. As used herein, torsion refers to both twisting and bending and torsional support refers to a structure providing resistance and support against twisting or bending.

First and second plates 100, 102 may be of any material and constructed in any manner that allows plates 100, 102 to establish a stable interface with adjacent tissue and that are safe for an implant recipient. For example, where implant 10 completely replaces a joint between two bones, plates 100, 102 may be constructed to establish a stable interface with adjacent bone. Establishing a stable interface may have both short and long term components. Specific structure may be included on plates 100, 102 to address each component. For example, plates 100, 102 may have structure ensuring that implant 10 remains in a desired location in the short term, following implantation. This structure may include, for example, protrusions 40, as illustrated in FIG. 4, such as, for example, teeth, ridges or serrations. Plates 100, 102 may also be constructed to interact with other fixation devices. For example, plates 100, 102 may have holes for receiving bone screws which may be used to affix them to the bone. Plates 100, 102 may also be constructed to facilitate implantation. For example, plates 100, 102 may include structure to mate with an implantation aid or other device, or to otherwise provide for safer and easier implantation. Similarly, plates 100, 102 may have structure ensuring that implant 10 remains in a desired location in the long term, and successfully interfaces with adjacent tissue. This structure may include, for example, a tissue ingrowth region 500 (See FIG. 4) that allows adjacent tissue to grow into the implant, forming a stable interface. Tissue ingrowth region 500 may include textured metal surfaces, porous surfaces, osteoinductive surfaces and osteoconductive surfaces. For example, tissue ingrowth region 500 may comprise sintered metal particles or a structure constructed by solid free form fabrication. As an alternate example, tissue ingrowth region 500 may include a textured metal surface textured by, for example, chemical etching or plasma spraying. According to one aspect of one embodiment, the tissue ingrowth region 500 may only include enough of plates 100, 102 to establish a stable interface with adjacent tissue, and plates 100, 102 may be predominantly solid.

It is to be appreciated that first and second plates 100, 102 may also be sized and shaped to establish a stable interface with adjacent tissue. For example, plates 100, 102 need not be flat and may be shaped to match the contour of adjacent tissue. For example, if the tissue adjacent to one of plates 100, 102 is concave or convex, the plate 100, 102 may be constructed with a curved shape to match the adjacent tissue of the joint. Similarly, plates 100, 102 need not be circular or oval as illustrated in FIGS. 1-4, rather, they may be any shape that allows establishment of a stable interface with adjacent tissue. Accordingly, implant 10 may have an irregular shape corresponding to an adjacent tissue such as a bone. For example, where implant 10 is used to replace an intervertebral disc, it may be shaped like a spinal disc, allowing it to fit easily between the vertebrae and to establish a stable interface therewith.

It is to be appreciated that first and second plates 100, 102 may be constructed of any material that is safe for a recipient of implant 10, and that allows a stable interface with adjacent tissue. For example, plates 100, 102 may be constructed of a material that is biocompatible, meaning that it is neither harmful to the health of an implant recipient, nor significantly damaged or degraded by the recipient's normal biology. Biocompatible materials include, for example, various metals and metal alloys, ceramic materials and synthetic materials, such as polymers. It is also to be appreciated that plates 100 may also be constructed of a material that is strong and durable enough to withstand the forces that may be placed upon it once installed in an implant recipient. For example, if implant 10 is used to replace a load bearing joint, the material for plates 100, 102 may be selected such that plates 100, 102 will not fail under stresses normally experienced by that joint. The ability of plates 100, 102 to withstand stresses also may be dependant on the shape and size of plates 100, 102 as well as their material of construction and, thus, it is to be appreciated that the size and shape of plates 100, 102 may also be considered when selecting a material. In one embodiment of an implant according to the application, plates 100, 102 are preferably constructed of titanium or a titanium alloy. A titanium alloy typically used in implants includes 6% aluminum and 4% vanadium by weight.

Referring now to FIGS. 1-3, axial support 200 may by constructed of any materials and in any manner that provides sufficient support and flexibility for a successful arthroplasty and is safe for the implant recipient. Axial support 200 may be constructed in a manner so that it provides support along an axis that is substantially perpendicular to the primary surfaces of plates 100, 102. This support may be sufficient for implant 10 to successfully bear loads that may be placed upon it. However, axial support 200 may also be constructed in a manner so that it provides flexibility so that it may successfully restore motion to a joint it replaces. For example, axial support 200 may be constructed as one or more struts or of first and second mating halves 202, 204 that provide sufficient support and flexibility to implant 10.

Where axial support 200 is constructed as one or more struts, the struts may be constructed to provide axial support to implant 10 and also to be flexible. For example, the struts may be relatively incompressible and may be arranged substantially perpendicular to plates 100, 102 as illustrated in FIG. 1. Accordingly, stress applied parallel to, and directly above, the struts will be resisted by each strut due to its incompressibility. Conversely, stresses not parallel to, or directly above, the struts will result in bending of the struts due to their flexibility.

In one embodiment of the implant of the application, the struts may be cables. Cables are typically relatively incompressible along their lengths and are also typically flexible. Accordingly, an axial support 200 constructed of one or more cables would provide support sufficient to replace the load bearing function of a joint while also allowing it to flex, resulting in a successful arthroplasty implant.

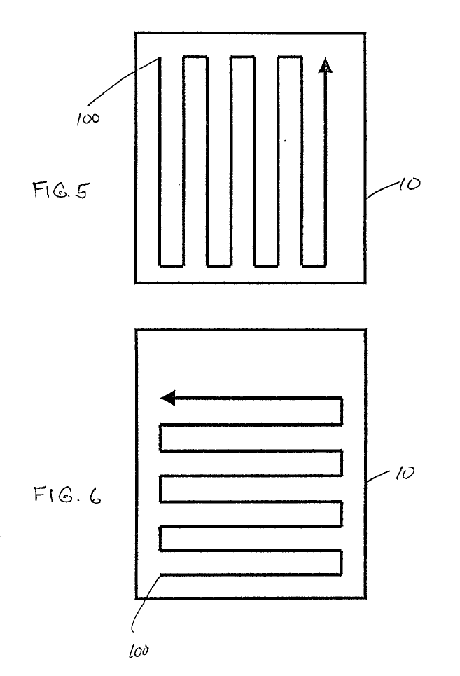

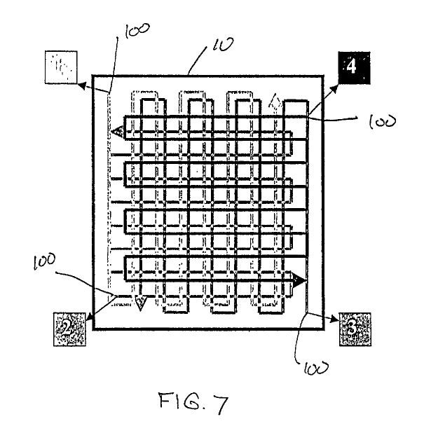

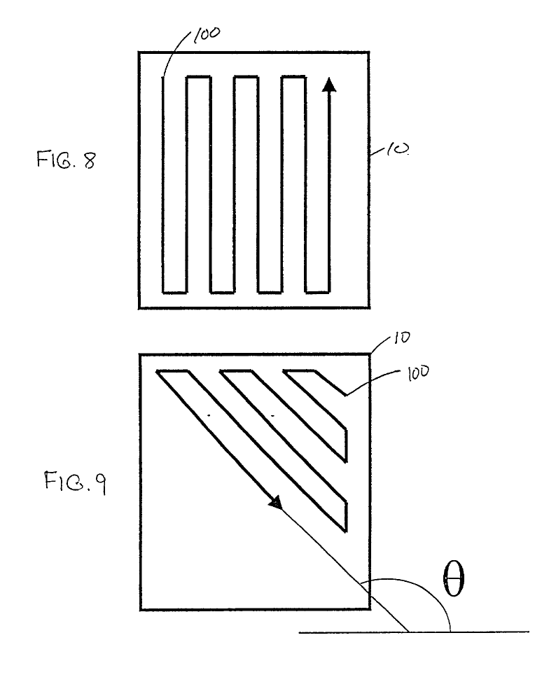

Axial support 200 may also comprise a single strut shaped to be flexible. For example, axial support 200 may comprise a strut that is tapered in a center region, as illustrated in FIGS. 2 and 4. Axial support 200 may also be designed to favor flexing in a particular direction. For example, as illustrated in FIGS. 14-22, axial support 200 may be wider in one dimension than the other. Where axial support 200 is wider in one dimension, it may increase the rigidity of axial support 200 in that direction, such that it provides adequate resistance to bending without torsional supports 300A, 300B, as will be described in more detail below. Although axial support 200 is illustrated in FIGS. 2 and 4 as having an oval cross-section, and in FIGS. 14-22 as having an oblong cross-section, any shape providing the desired support and flexibility for axial support 200 may be used.

Where axial support 200 is constructed of mating halves 202, 204, it may be constructed to provide sufficient support and flexibility for a successful arthroplasty and to provide a stable connection between halves 202, 204. For example, halves 202, 204 may be constructed to provide a stable connection and such that they may not slip off of one another or otherwise become detached from one another. Halves 202, 204 may also be constructed such that axial support 200 is still flexible. For example, halves 202, 204 may form a joint capable of articulation, such as a ball and socket joint as illustrated in FIG. 3, that may be sufficiently stable to withstand stresses typically applied to a joint, yet that will not detach, and that is articulable to provide flexibility to axial support 200.

It is to be appreciated that axial support 200 may be located anywhere between plates 100, 102 to provide support and flexibility at any portion of plates 100, 102. The location of axial support 200 may depend on the nature of implant 10 and the type of joint being replaced. Typically, axial support 200 will be located at the point about which the joint it replaces normally pivots. This may be near the center of plates 100, 102, however, it need not be. Axial support 200 may be connected to the plates 100, 102 by any method that will maintain it in a proper location and is not subject to failure. For example, axial support 200 may be welded to plates 100, 102, or it may be unitarily formed with plates 100, 102 such as described above using the LENS method.

It is to be appreciated that axial support 200 may be constructed of any material that is safe for a recipient of implant 10 and that can withstand the stresses and friction that will be placed upon it. For example, axial support 200 may be constructed of a material that is biocompatible. Axial support 200 may also be constructed of a material that is strong and durable enough to withstand the forces that may be placed upon it once installed in an implant recipient. For example, if implant 10 is used to replace a load bearing joint, the material axial support 200 is constructed from may be selected such that axial support 200 does not fail under stresses normally experienced by that joint. The ability of axial support 200 to withstand stresses also may be dependant on the shape and size of axial support 200 as well as its material of construction and, thus, size and shape of axial support 200 may also be considered when selecting a material. Where axial support 200 also comprises an articulating joint, the material that axial support 200 is constructed from may be selected to be resistant to frictional wear. In one embodiment of an implant according to the application, axial support 200 is preferably constructed of titanium or a titanium alloy.

Where axial support 200 is a single piece of material it may be fabricated from a polymer or composite of a polymer and other reinforcement. Typical reinforcements include but are not limited to carbon or glass fibers, either continuous or chopped in form. Other reinforcements may be thin sheets of metals that are laminated together using polymers as adhesives. The size, shape, orientation and amount of these reinforcements may be such that the mechanical properties of axial support 200 can be engineered to meet the flexibility and strength requirements.

It is to be appreciated that axial support 200 may be constructed by any method that will provide desired properties and long life. The method of construction of axial support 200 may vary with the material from which it is constructed. For example, in some embodiments axial support 200 may be cast, machined or otherwise formed and then attached to plates 100, 102, such as by welding. However, one possible disadvantage of manufacturing axial support 200 separately from plates 100, 102, in other words other than as a unitary structure, is that the points of attachment may weaken and be subject to fatigue and possibly failure. Furthermore, if the strut is bent or twisted once formed, this deformation may result in micro-cracking and other structural degradation. Accordingly, in one embodiment of an implant according to the application, it is preferred that axial support 200 is unitarily formed with plates 100, 102. For example, plates 100, 102 and axial support 200 may be formed by the LENS method, and particularly by the method of the present application. For example, implant 10 formed by the LENS method may be comprised of solid metal and may be formed in the exact shape desired, eliminating the need to attach axial support 200 to plates 100, 102 or to twist or bend axial support 200.

It is also to be appreciated that torsional supports 300A, 300B may be constructed of any material and in any manner that provides sufficient resistance to bending and torsion of implant 10 to allow implant 10 to provide the torsional support function of the joint replaced, but that is also sufficiently flexible to allow implant 10 to bend or turn where desired, such as in twisting or bending of a spinal implant according to the normal movement of the spinal column. Torsional supports may also be constructed of a material and in a manner that is safe for a recipient of implant 10. Torsional supports 300A, 300B also may be constructed in a manner that provides sufficient resistance to bending and torsion to allow implant 10 to support surrounding tissue and prevent injury due to excessive bending or torsion. For example, axial support 200 may be flexible and may not provide sufficient resistance to bending or torsion. Accordingly, if torsional supports 300A, 300B do not provide sufficient resistance to bending and rotation, implant 10 may allow over-rotation or excessive bending of a joint, potentially resulting in injury. For example, where implant 10 is used to replace a spinal disc, over-rotation or excessive bending could result in pain or damage to the nerves of the spinal column. Accordingly, torsional supports 300A, 300B preferably provide some resistance to bending and may also allow torsional supports 300A, 300B to perform some of the shock absorbing function that the replaced joint had.

While torsional support 300A, 300B may provide some resistance to torsion or bending, torsional supports 300A, 300B are preferably provided so that this resistance is not so great that desired motion of the joint is lost. For example, it may be desired to restore a full range of motion to a joint replaced by implant 10, and torsional support 300A, 300B may have a degree of resistance to torsion and bending that limits the implant to the range of motion of the original joint, but not more than this.

In one embodiment of an implant of the application, torsional support 300A, 300B may be comprised of one or more struts to provide resistance to torsion and bending while still allowing desired motion. For example, one or more struts may extend from first plate 100 to second plate 102. The struts may be arranged such that, unlike some embodiments of axial support 200, pressure directly against plates 100, 102 at the ends of the struts will not be resisted by the struts due to their incompressibility, but rather, the struts act as a spring. For example, the struts may be curved, or the top and bottom of these struts may not be directly above one another. As illustrated in FIGS. 1-4, struts may curve around some portion of axial support 200 while extending between plates 100, 102, providing simultaneous flexibility and resistance both to torsion and to bending of implant 10 around axial support 200.

It is to be appreciated that where even resistance to bending and torsion is desired for implant 10, torsional supports 300A, 300B may be symmetrical. For example, where there are two torsional supports 300A, 300B, the torsional supports may be mirror images of one another as illustrated in FIG. 2, or where there are more than two torsional supports 300A, 300B, they may be equally spaced around axial support 200. Where even resistance to bending and torsion is not desired, torsional supports 300A, 300B may be asymmetrical to provide more resistance where more resistance is desired, or additional torsional supports may be used at these locations.

It is to be appreciated that torsional supports 300A, 300B may be constructed by any method that will provide desired properties and long life. For example, torsional supports may be cast, machined or otherwise formed and then attached to plates 100, 102, such as by welding. However, one possible disadvantage of manufacturing torsional supports 300A, 300B separately from plates 100, 102, in other words other than as a unitary structure, is that the points of attachment may weaken and be subject to fatigue and possibly failure. Furthermore, if the struts are bent or twisted once formed, this deformation may result in micro-cracking and other structural degradation. Accordingly, in one embodiment of an implant according to the application, it is preferred that torsional supports 300A, 300B are unitarily formed with plates 100, 102. For example, plates 100, 102 and torsional support 300A, 300B may be formed by the LENS method, and particularly by the method of the present application. For example, implant 10 formed by the LENS method may be comprised of solid metal and may be formed in the exact shape desired, eliminating the need to attach torsional support 300A, 300B to plates 100, 102 or to twist or bend torsional support 300A, 300B.

It is to be appreciated that torsional supports 300A, 300B may be constructed of any material that is safe for a recipient of implant 10, that can withstand the stresses that will be placed upon it, and that also has sufficient flexibility to allow desired motion of implant 10. For example, torsional supports 300A, 300B may be constructed of a material that is biocompatible. Torsional support 300A, 300B may also be constructed of a material that is strong and durable enough to withstand the forces that may be placed upon it once installed in an implant recipient. For example, torsional supports 300A, 300B may be constructed from a material such that torsional supports 300A, 300B may not fail under stress or repeated bending normally experienced by a joint it replaces. The ability of torsional supports 300A, 300B to withstand stresses also may be dependant on the shape and size of torsional supports 300A, 300B as well as their material and method of construction. Thus, size and shape of torsional supports 300A, 300B may also be considered when selecting a material. In one embodiment of an implant of the application, torsional supports 300A, 300B are preferably formed of a metal, and this metal is the same as that used to form plates 100, 102 to facilitate construction by the LENS method. Accordingly, it is also preferred that torsional supports 300A, 300B are constructed of titanium or a titanium alloy.

In another embodiment axial support 200 and torsional support 300A 300B may be combined into a single flexible support 400 or either of the axial or torsional supports may be eliminated. Support 400 is briefly described above in connection with FIGS. 14-22. In addition to providing resistance to bending, for example as in the embodiment illustrated in FIGS. 14 and 15, such a support may also provide shock absorbing capability that may have been present in the joint being replaced. For example, support 400 may be compressible in the axial direction. Examples of supports 400 compressible in the axial direction are illustrated in FIGS. 16-22. In some embodiments, such supports 400 may have a single connection to first plate 100 and a single connection to second plate 102. For example, support 400 may be constructed similarly to some embodiments of torsional support 300A, 300B in that support 400 may be arranged such that pressure applied against plates 100, 102 at the ends of support 400 will not be resisted by support 400 due to its incompressibility, but rather, support 400 acts as a spring. For example, support 400 may be curved, or the top and bottom of support 400 may not be directly above one another, as illustrated in FIGS. 16-22.

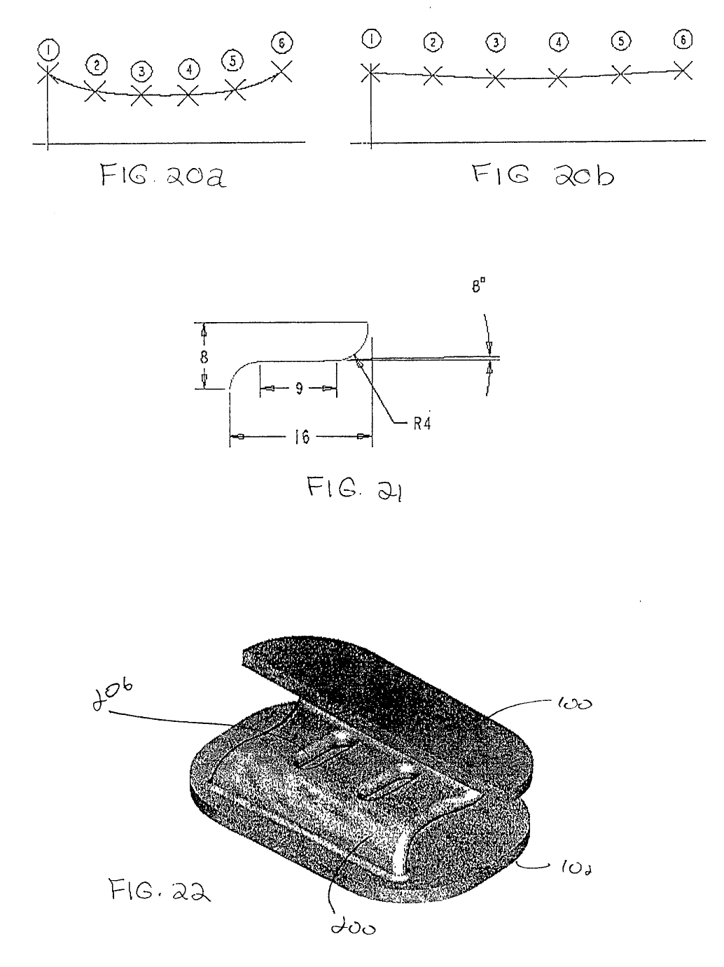

In one embodiment, implant 10 mimics the movement of a natural disc, including shock absorption, torsion and flexion, using primarily or exclusively support 400. For example, support 400 may comprise a swept structure that connects plates 100 and 102 at its ends, as illustrated in FIGS. 16-22. Such a swept support may allow shock absorption, torsion and flexion and may be shaped to adjust these properties. For example, support 400 may be shaped to distribute stress evenly over its length, leading to improved flexion and durability. For example, as illustrated in FIGS. 19-21, swept support 400 may vary in cross section. Exemplary uniform and varied cross-section embodiments may be seen by comparing FIGS. 17 and 20. FIG. 17 is a graph of the cubic spline of the width and thickness of support 400 as illustrated in FIG. 16 at six evenly spaced points along its length and shows that these properties do not vary as a function of distance along swept support 400. The values of width and thickness associated with each of these points in one example embodiment is illustrated in Table 2.

TABLE 2

| | % along | | |
|---|---|---|---|
| Point | support | Width | Thickness |
| 1 | 0% | 32 mm | 2.3 mm |
| 2 | 20% | 32 mm | 2.3 mm |
| 3 | 40% | 32 mm | 2.3 mm |
| 4 | 60% | 32 mm | 2.3 mm |
| 5 | 80% | 32 mm | 2.3 mm |
| 6 | 100% | 32 mm | 2.3 mm |

FIGS. 20a and 20b are graphs of the cubic spline of the width (left) and thickness (right) of support 400 as illustrated in FIG. 19 at six evenly spaced points along its length showing that these properties vary along the length of swept support 400 and are at their lowest at substantially the center of support 400. The values of width and thickness associated with each of these points in one example embodiment is illustrated in Table 3.

TABLE 3

| | % along | | |
|---|---|---|---|
| Point | support | Width | Thickness |
| 1 | 0% | 32 mm | 2.3 mm |
| 2 | 20% | 27 mm | 2.2 mm |
| 3 | 40% | 24.5 mm | 2.1 mm |
| 4 | 60% | 24.5 mm | 2.1 mm |
| 5 | 80% | 27 mm | 2.2 mm |
| 6 | 100% | 32 mm | 2.3 mm |

Decreasing the size of support 400 near its center is one way of distributing stress evenly along its length.

Features of swept support 400 other than the cross-section, may also be varied. For example, while a central portion of support 400 is illustrated in FIGS. 16-22 as substantially parallel to plates 100, 102, which are, in turn, substantially parallel to one another, these angles may be varied. It is to be understood that according to this application, by indicating that surfaces are substantially parallel, it is meant that surfaces are not more than 30° from parallel, and, in preferred cases, are less than 10° and more preferably, less than 5° from parallel. In some embodiments, such as those illustrated in FIGS. 18-21, the center region of support 400 may be less than 1°, such as 0.8° off parallel from one of plates 100, 102. As has already been discussed, plates 100 and 102 may also vary from parallel, as can be seen in FIGS. 14 and 16, depending on the needs of a particular implant application.

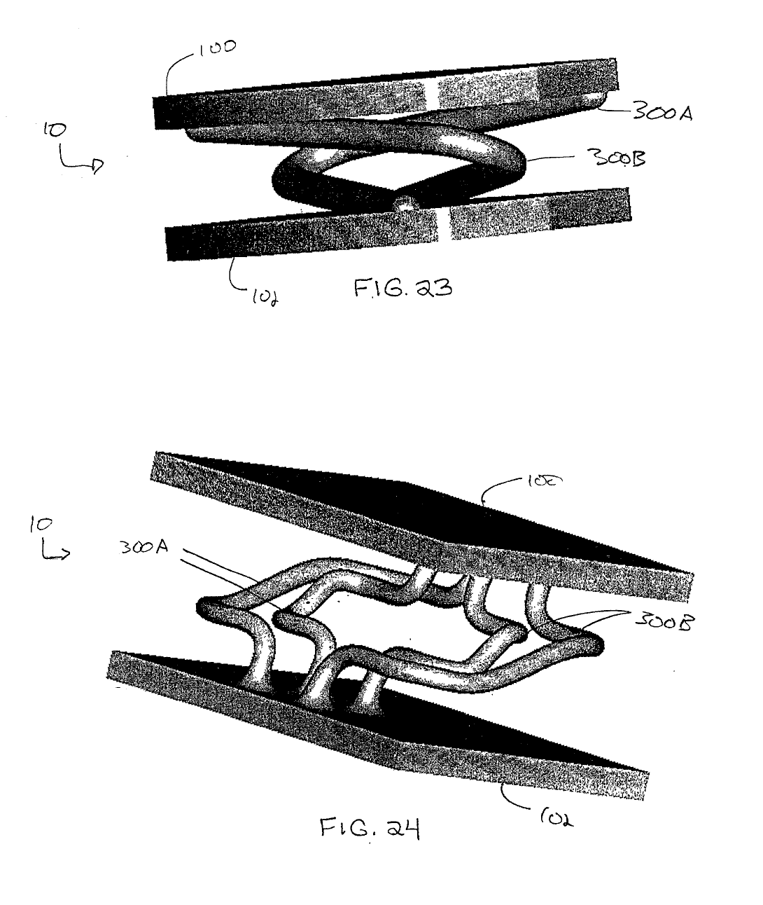

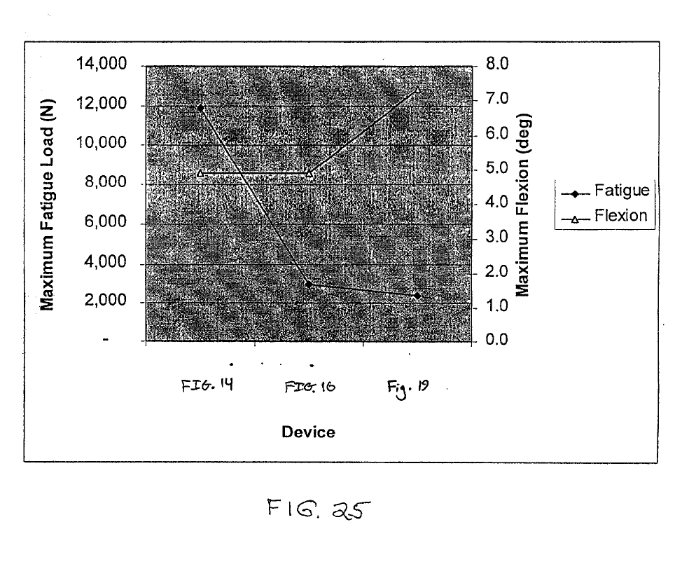

According to one aspect of one embodiment of an implant 10, where support 400 is swept, it need not be constructed as a single solid piece. For example, support 400 may include one or more holes 206, as illustrated in FIG. 22. Such holes 206 may be located near the center of swept support 400 such that they facilitate distribution of any load evenly along its length. In an alternate example, swept support 400 comprises multiple supports 400 (not illustrated). In some embodiments, such those illustrated in FIGS. 23-24, modified torsional supports 300A, 300B may act as both torsional and axial supports 400.

As discussed above, the design of an orthopedic implant is a function of the role it is intended to fill. For example, cervical disc replacements may require more flexibility and less load bearing capability than lumbar disc replacements, which, in turn, may require more flexibility and less load bearing capability than an ankle joint replacing implant. FIG. 25 illustrates some of the tradeoffs that may exist in the design of an orthopedic implant, comparing the maximum fatigue load and maximum flexion of the supports of FIGS. 14, 16 and 19. The dimensions of the supports of the embodiments of FIGS. 16 and 19 are given in Tables 2 and 3. The dimensions of the support of the embodiment of FIG. 14 was, with reference to FIG. 15, 38 millimeters wide and 1.6 millimeters thick. It should be understood that these dimensions are intended by way of example only. As illustrated in the FIG. 25, the vertical support of FIG. 14 has the greatest load carrying capacity, but also the least flexibility, while the swept support having varying cross section of FIG. 19 has the least load carrying capacity and the greatest flexibility. The swept, constant cross-section embodiment of FIG. 16 falls between these two extremes. Accordingly it can be seen that the design of an implant is often an optimization process dictated by the particular needs of the implant.

As support 400 may fill the same roles as axial support 200 and torsional supports 300A, 300B, it my be constructed with the materials or methods described above for either of these structures, some combination of these methods and materials, or entirely different methods and materials that allow it to perform the desired functions. Preferably, support 400 is constructed by the LENS process, and particularly by the method of the application as has been previously described.

One method of designing an orthopedic implant according to the application includes loading a proposed implant with an axial load in the physiologic range for the joint to be replaced, to test for failure. If there is a failure, the method further includes altering the geometry of the implant, such as orientation and cross section of the support, to withstand the axial load. Once geometry is attained that will support the axial load, the proposed implant may be loaded with a target flexion/extension bending load and the displacement measured. If necessary, the geometry of the implant, such as the orientation and cross section of axial support may again be altered to withstand the bending load. These steps may be iterated as desired to achieve the desired level of displacement under a target flexion/extension bending load. This method could be repeated in other dimensions until an implant having the desired properties in every dimension is constructed.

An implant, material and method of manufacture of the present application will be further illustrated by the following examples, which are intended to be illustrative in nature and not considered as limiting to the scope of the application.

### EXAMPLES

### Example 1

One suitable construction of an implant having a shape and design substantially in accordance with the present application is provided by the following combination of elements.

An implant 10 to be used in a spinal arthroplasty includes a first plate 100 and a second plate 102. Plates 100, 102 are substantially oval and planar and are sized to fit within a human spinal column in a space previously occupied by a disc. The outer planar surfaces of plates 100, 102 are provided with protrusions 400 consisting of teeth and a tissue ingrowth region 500 consisting of a textured surface.

Implant 10 also includes an axial support 200, between, and connecting, plates 100, 102. Axial support 200 is oriented in the center of plates 100, 102 and includes a cable incorporated at both ends to plates 100, 102. Implant 10 further includes two torsional supports 300A, 300B. Torsional supports 300A, 300B are unitarily formed with plates 100, 102 and curve around axial support 200 such that the first end of each of torsional supports 300A, 300B is not directly across from the second end of each of torsional supports 300A, 300B. Torsional supports 300A, 300B are mirror images of one another. Implant 10 is constructed of an alloy of titanium with 6% aluminum and 4% vanadium by weight.

Implant 10 may have an outer envelope of approximately 20 mm in an anterior/posterior direction, 30 mm in a lateral direction, and 12 to 15 mm in height. The overall shape mimics that of a vertebral disc and is roughly kidney shaped. The size of torsional supports 300A, 300B are dependant on the material selected, but may be about 5 mm or less in diameter. Axial support 200 may be about 10 mm in diameter.

### Examples 2-7

In order to determine what values for Φ produce acceptable build height and preferred microstructure, a series of six examples, numbered 2-7, were made with varied parameters. For all six examples a titanium alloy containing 6% aluminum and 4% vanadium was used. The article constructed in each example was 0.0127 m×0.0127 m×0.019 m. The build parameters and resultant microstructure are shown in Table 1.

TABLE 1

| | | | | | | Preferred | |
|---|---|---|---|---|---|---|---|
| | | | Powder | | | Micro- | |
| | Laser | Speed | Feed Rate | Φ | | structure | In Method |
| Example | Power (J/s) | (m/s) | (kg/s) | (× 10⁶) | Microstructure | range | Range |
| 2 | 342 | 0.0254 | 0.00013 | 9.8 | Refined β grain structure | ✓ | ✓ |
| | | | | | (comprising 71.3%) | | |
| 3 | 415 | 0.03387 | 0.000152 | 10.76 | Refined β grain structure | ✓ | ✓ |
| | | | | | (comprising 76.9%) | | |
| 4 | 342 | 0.0254 | 0.00013 | 9.9 | Refined β grain structure | ✓ | ✓ |
| | | | | | (comprising 65.2%) | | |
| 5 | 342 | 0.0339 | 0.00016 | 9.3 | Large columnar β grain structure, | X | ✓ |
| | | | | | (refined β grain structure | | |
| | | | | | comprises 31.7%) | | |
| 6 | 558 | 0.0254 | 0.00013 | 15.9 | Large columnar β grain structure, | X | ✓ |
| | | | | | (refined β grain structure | | |
| | | | | | comprises 18.1%) | | |
| 7 | 342 | 0.0339 | 0.00013 | 7.5 | Large columnar β grain structure, | X | ✓ |
| | | | | | (refined β grain structure | | |
| | | | | | comprises 9.2%) | | |

For purposes of this example, whether a sample fell into the preferred microstructure range was determined based on whether at least half of grain structure was in oriented layers as opposed to long grains extending through multiple layers. It should be appreciated that this criteria was selected by way of illustration and that, in some embodiments, having 10 or 25% refined microstructure may be sufficient for certain applications, while in others 70, 75 or 80% may be preferred.

Referring to Table 1, it is clear that values for Φ of between about 9.5×10⁶ and about 11×10⁶ are in the preferred range of microstructure while values of Φ of between about 5.0×10⁶ and about 20×10⁶ provide acceptable build height (δ).

### Example 8

In order to determine the yield load, strain data and displacement of the embodiment of the application illustrated in FIGS. 19-21, the implant was tested under axial compressive loading and compressive loading 45 degrees from axial. The implant was constructed of titanium alloy containing 6% aluminum and 4% vanadium using the LENS process and had the preferred microstructure of the invention. The dimensions of the article tested were identical to that given in Table 3, except that the thickness of the swept structure was constant along its length at 2.1 mm.

Under axial testing, none of three specimens failed at loads up to 8900 N, but did incur permanent deformation on the order of 1.3 mm, or 11% of the original device height. Parts showed yielding at approximately 3560 N, exceeding the expected 3000 N yield resistance.

Under testing 45 degrees from axial, none of three specimens failed at loads up to 8900 N, but did incur permanent deformation on the order of 1.0 mm along the axis the load was applied. Parts showed yielding at approximately 5340 N, exceeding the expected 3800 N yield resistance.

Strain data from this testing indicates that the titanium alloy having the preferred microstructure had a yield strength of about 936 to 1,111 MPa as compared to standard annealed versions of this alloy having a yield strength of about 880 MPa.

The device of the present application demonstrated improved performance based upon its microstructure and withstood loads far greater than those capable of causing failure to a vertebral body (approximately 1,500N). Accordingly, the device would not be a failure point in a traumatic loading condition.

Having thus described at least one preferred embodiment of the implant and method of the application, various alterations, modifications and improvements will readily occur to those skilled in the art. Such alterations, modifications and improvements are intended to be part of the disclosure and to be within the spirit and scope of the application. Accordingly, the foregoing description is by way of example only and is limited only as defined in the following claims and equivalents thereto.

What is claimed is:

1. An orthopedic implant, comprising:
a first plate;
a second plate; and
a flexible support having a single connection to the first plate and a single connection to the second plate;
wherein the first plate, the second plate and the flexible support are unitarily formed.

2. The orthopedic implant of claim 1, wherein the flexible support and the first and second plates are constructed of the same material.

3. The orthopedic implant of claim 2, wherein the material is biocompatible.

4. The orthopedic implant of claim 3, wherein the material comprises titanium.

5. The orthopedic implant of claim 1, wherein in a first position the flexible support is substantially perpendicular to the first plate and the second plate and the first plate and the second plate are substantially parallel to one another.

6. The orthopedic implant of claim 1, wherein the first plate and second plate are substantially planar.

7. The orthopedic implant of claim 1, wherein the first plate and the second plate comprise one or more protrusions.

8. The orthopedic implant of claim 1, wherein the first plate and the second plate comprise openings sized and adapted for insertion of a fixation device.

9. The orthopedic implant of claim 1, wherein the first plate and the second plate each comprise a tissue ingrowth region.

10. The orthopedic implant of claim 9, wherein the tissue ingrowth region is porous.

11. The orthopedic implant of claim 9, wherein the tissue ingrowth region comprises a textured metal surface.

12. The orthopedic implant of claim 1, wherein the flexible support varies in cross-section along a length of the flexible support.

13. The orthopedic implant of claim 1, comprising no more than one flexible support.

14. The orthopedic implant of claim 1, wherein the single connection to the first plate is axially displaced from the single connection to the section plate.

15. An orthopedic implant, comprising:
a first plate;
a second plate;
and a flexible support comprising a connection to the first plate, a connection to the second plate and a cross section varying along a length of the flexible support.

16. The orthopedic implant of claim 15, wherein the flexible support comprises no more than one connection to the first plate and one connection to the second plate.

17. An orthopedic implant produced by a method comprising directing a stream including particulate material in a pattern corresponding to at least a portion of a structure of the orthopedic implant and fusing at least a portion of the particulate material with a laser.

18. An article, comprising:
a metallic component comprising layers including grains of precipitated phase wherein a majority of the grains of precipitated phase in each layer are oriented in substantially two opposed directions and the two opposed directions are substantially different from a direction of orientation of grains in an adjacent layer.

19. An orthopedic implant, comprising:
an outer wall; and
a flexible support connected to the outer wall in at least two locations.
